(19) United States
(12) Patent Application Publication
(10) Pub. No.: US 2026/0060402 A1
(43) Pub. Date: Mar. 5, 2026

(54) REMOVABLE NAIL COATINGS AND COMPOSITIONS AND METHODS OF PREPARING THEREOF

(71) Applicant: Liqwd, Inc., Santa Barbara, CA (US)

(72) Inventors: Craig J. Hawker, Santa Barbara, CA (US); Christopher M. Bates, Santa Barbara, CA (US); Javier Read de Alaniz, Santa Barbara, CA (US); Morgan Bates, Santa Barbara, CA (US); Kaitlin Albanese, Santa Barbara, CA (US)

(21) Appl. No.: 19/318,837

(22) Filed: Sep. 4, 2025

Related U.S. Application Data
(60) Provisional application No. 63/691,107, filed on Sep. 5, 2024.

(51) Int. Cl. A45D 29/00; A61K 8/37; A61K 8/49; A61K 8/55; A61Q 3/02

(52) U.S. Cl. CPC A45D 29/00; A61K 8/37; A61K 8/4986; A61K 8/55; A61Q 3/02

(57) ABSTRACT

Methods and compositions for preparing multilayer nail coatings that enable facile removal of nail enhancements formed thereon are described herein. The nail enhancements become removable upon exposure to a suitable removal stimulus.

### CROSS-REFERENCED TO RELATED APPLICATIONS

This application claims the benefit of and priority to U.S. Provisional Application No. 63/691,107, filed Sep. 5, 2024, which is hereby incorporated herein by reference in its entirety.

### FIELD OF THE INVENTION

The disclosed invention is generally in the field of cosmetics, particularly in the field of removable nail enhancements.

### BACKGROUND OF THE INVENTION

Commercially available nail enhancements, which are formed on nails from commercially available nail products, may be classified into different categories based on their composition, chemistry, and method of use, for example: (1) nail polishes, also known as lacquers, varnishes, or enamels: (2) gels, including acrylics; (3) dip powder systems; and (4) artificial nails such as pieces of shaped plastic that can be attached to a nail using gel, dip, or other types of adhesives; as well as combinations thereof. Nail polishes typically include various solid components that are dissolved and/or suspended in non-reactive solvent(s). Upon application and drying, the solids deposit on the nail surface as a clear, translucent, or colored film. Nail polishes are easily scratched and are readily removable with solvent, usually within one minute and if not removed as described, will chip or peel from the natural nail in one to five days. The ability to easily remove nail polishes when desired is a benefit for users, but the lack of mechanical robustness and longevity is a major downside.

In contrast to nail polishes, commercially available gel products applied by a salon or purchased in at-home kits last for up to two weeks without damage. This improved longevity has made gel products quite popular, and they are available in a variety of styles. The reason gel products are more mechanically robust is related to the very different mechanism by which gel manicures solidify compared to conventional nail polishes. Each layer is applied to the nail, for example, by painting it on with a brush, and then cured under light, e.g., an ultraviolet (UV) light or light-emitting diode (LED). The curing process results in solidification of the as-applied liquid layer via a polymerization reaction that causes crosslinking, yielding a coating that is significantly stronger and longer-lasting than conventional nail polishes such as nitrocellulose-based formulations. Procedurally, this process is usually repeated several times per nail. For example, a common process of applying gel products involves: (1) first removing any existing product, optionally with additional nail-surface preparation such as buffing, grinding, or wiping with a solvent such as isopropanol; (2) applying a base coat; (3) optionally applying one or more color coats to change the appearance of the product; and (4) applying a top coat to create an appealing surface finish. After steps 2, 3, and 4, each as-deposited liquid layer is exposed to light, e.g., a UV lamp and/or LED, to solidify it before moving on to the next one. Sometimes, fingerless gloves are worn to keep UV or LED rays off the hands.

Chemically, gel products are mixtures of monomers, oligomers, and/or polymers, often with additional additives, all of which tune the formulation properties in the liquid state, its reactivity, and the corresponding properties in the solid state after crosslinking, Products such as UV gel nails usually include acrylate, methacrylate, acrylamide, and/or methacrylamide monomers, oligomers, and/or polymers, or other components that undergo a radical-mediated polymerization as initiated by one or more photoinitiators present in the formulation. A related type of product known as acrylic liquid-and-powder nails also involves the solidification of similar monomers, oligomers, and/or polymers by a free-radical polymerization process, but in this case, initiation is induced by thermal initiator(s) such as peroxides present in the powder when it comes in contact with the liquid, rather than a photoinitiator creating radicals under UV or LED light.

Many users enjoy gel products because they last longer than nail polishes without chipping or lifting. However, this improved longevity makes them more difficult to remove from the nail. If a user desires a different nail enhancement or simply to remove the gel product, often times grinding and/or soaking in a solvent such as acetone for an extended period of time (e.g., 10 to 30 minutes) is required. Both of these removal techniques can damage a user's nails, making them undesirable and potentially unhealthy but essentially unavoidable with gel products today.

An even longer-lasting nail product than gels and acrylics gaining popularity is known as dip powder. Dip powder systems involve multiple components in a kit that are applied sequentially. First, the surface of the nail can be prepared as desired by washing, buffing, grinding, dehydrating, or the like. Second, a “base” coat is applied in the liquid (or viscous liquid) state. Third, before the applied base coat fully solidifies, the nail is quickly dipped into a solid powder that sticks as a separate layer to the surface of the base coat. Any excess powder can be lightly brushed off as needed. There are different types of powders that can be selected based on the user's desired appearance for the nail, for example, a wide variety of colors and styles such as sparkles are commercially available. This process of applying the base coat followed quickly by dipping the wet surface into a powder can be repeated multiple times, and the same or different powders can be used in each repetition. Various other products can be included in the kit and applied before, during, or after different parts of the process, for example, a separate “activator” solution might be used after the last powder step (or, alternatively, after each powder step) to promote more complete and/or faster solidification of the base layer(s). Often times the very last step of the process involves applying a “top” coat to create an appealing visual look and tactile sensation. Buffing may also be used to smooth the surface at various points in the overall application process.

Dip powder systems exploit a very different type of chemistry than the aforementioned nail polishes and gel/acrylic products. Although the base and top coat layers of a dip-powder system may contain multiple components such as additives like poly(methyl methacrylate), the key ingredients in each are cyanoacrylate-based monomers, oligomers, and/or polymers. Cyanoacrylates can be applied to the nail or dip-powder-coated layers as a liquid, but the as-applied liquid spontaneously solidifies within minutes without any additional stimulus because the small amount of water present everywhere (e.g., in air, on the nail, in any underlying layers) initiates anionic polymerization. (The aforementioned “activator” solution is an accelerator such as a tertiary aryl amine that also promotes anionic polymerization.) This chemistry is analogous to super glue, and like super glue, these coatings are very mechanically robust, strong, and long-lasting. Dip powder systems are therefore even more difficult to remove than gel products. Removal often involves aggressive mechanical grinding and abrasion plus swelling with solvent for extended periods of time, which as mentioned above, can damage a user's nails. Such damage may limit the frequency at which users can change dip powder nail products or even cause a temporary pause in the use of nail-enhancement products to provide time for the nail to heal and regenerate after removing one product but before applying another.

Therefore, while gels, acrylics, and dip powder nail enhancements are popular options for achieving long-lasting and aesthetically pleasing manicures and pedicures, their removal remains a significant challenge with the potential to damage the nail bed and cause discomfort, bleeding, infection, nail loss, or prolonged health issues.

Thus, there remains a need for providing nail coatings that could address and overcome the challenges associated with removing commercially available nail enhancements, including damage to a user's nails,

Accordingly, it is an object of the invention to provide nail coatings that enable nail enhancements to be removed more easily when removal is desired.

It is a further object of the invention to provide methods for preparing such nail coatings and removable nail enhancements thereon.

It is yet a further object of the invention to provide compositions for forming such nail coatings.

### BRIEF SUMMARY OF THE INVENTION

Described herein are methods for forming multilayer nail coatings that allow for facile removal of nail enhancements present on a nail, as well as compositions for forming such multilayer nail coatings.

In one non-limiting instance, a method of forming a multilayer nail coating and a nail enhancement thereon includes the steps of:
(a) applying a nail primer layer to the nail;
(b) contacting the primer layer with a nail powder composition to form a nail powder layer thereon;
(c) optionally removing any excess of the nail powder composition, such as by tapping, brushing, wiping, and/or other physical means, from the coated nail;
(d) optionally exposing the nail primer layer coated with the nail powder layer to a stimulus, such as light or heat, for a sufficient period of time to induce at least partial curing of the nail primer layer;
(e) applying one or more nail products to the multilayer nail coating formed from steps (a)-(d) to form the nail enhancement thereon;
wherein the nail powder composition comprises:
   a plurality of particles wherein at least a portion of the particles can undergo a phase and/or shape transition upon exposure to a removal stimulus, such as a thermal stimulus, an electromagnetic stimulus, or a combination thereof.

The nail powder composition typically contains a plurality of particles, and at least a portion of the particles undergo a phase and/or shape transition upon exposure to a suitable removal stimulus, such as a thermal stimulus, an electromagnetic stimulus, or a combination thereof. In some instances, the at least a portion of the particles that undergo a phase and/or shape transition upon exposure to a stimulus, such as a thermal stimulus, an electromagnetic stimulus, or a combination thereof, are formed of primary particles having secondary particles, which are thermally expandable, contained within.

The multilayer nail coatings can be used in various applications in the nail care industry. The nail enhancements overlying the multilayer nail coatings are at least partially removable by exposure to a suitable removal stimulus, as described in the removal methods detailed below. For example, in one non-limiting instance, a method of removing a nail enhancement formed by any of the methods described herein includes the steps of:
(a′) applying a removal stimulus for an effective amount of time to a multilayer nail coating and nail enhancement thereon;
wherein when the removal stimulus is applied for the effective amount of time, it induces the plurality of particles of the multilayer nail coating to undergo a thermally induced phase and/or shape transition causing debonding, delamination, detachment, and/or a change in density that renders the nail enhancement at least partially removable, as compared to prior to the application of the removal stimulus.

In some instances, the removal stimulus is a thermal stimulus, photochemical stimulus, photothermal stimulus, or electromagnetic stimulus, or a combination thereof. In some instances, the method further includes applying an induction responsive nail heating composition directly on top of the nail enhancement prior to step (a'), where the induction responsive nail heating composition is able to generate a thermal removal stimulus.

Kits are also described for nail care applications, such as for forming the multilayer nail coatings and applying the nail enhancements (such as commercial nail products) thereon, which are removable on-demand by way of application of a suitable removal stimulus.

### BRIEF DESCRIPTION OF THE DRAWINGS

The accompanying drawings illustrate several embodiments of the disclosed compositions and methods thereof which together with the description, serve to explain the principles of the disclosed methods and compositions.

FIG. 1A shows a non-limiting illustration of a multilayer nail coating 100 including a nail primer layer 110 and a nail powder layer 120, where the multilayer nail coating is formed on a nail and a nail enhancement 130 is present on the multilayer nail coating.

FIG. 1B shows a non-limiting illustration of a multilayer nail coating 100′ including two nail primer layers 110′and two nail powder layers 120′, where the multilayer nail coating, which forms a stack, is on a nail and a nail enhancement 130′ is present on the multilayer nail coating.

FIG. 2 shows a non-limiting scheme of a method of forming a multilayer nail coating and a nail enhancement thereon.

FIG. 3 is a photograph of a commercially available nail gel product applied on top of a multilayer nail coating where the nail gel product detaches from the underlying multilayer nail coating and from the glass slide.

FIG. 4 is a photograph of a commercially available nail dip powder system applied on top of a multilayer nail coating where the nail product detaches from the underlying multilayer nail coating and from the glass slide.

### DETAILED DESCRIPTION OF THE INVENTION

Methods of forming multilayer nail coatings which allow for facile removal of nail enhancements thereon, and compositions for forming such multilayer nail coatings, are described herein.

### I. Definitions

The term “cosmetically acceptable,” as used herein, refers to compositions, formulations, or components thereof that are suitable for use in contact with human keratinous tissue (such as nail or skin) without undue toxicity, incompatibility, instability, allergic response, and the like.

The term “coating” refers to a deposit formed on at least a portion of a surface of a keratin material, such as a nail surface, where the deposit adheres to the keratin material.

The term “powder” refers to a discrete particulate form, such as a collection of particles, and is not considered a single macroscopic form.

The term “removable” refers to the ability of a nail enhancement (such as formed from one or more nail products) to be removed, detached, or delaminated completely or partially from a nail surface onto which the nail enhancement has been formed. This is performed while causing no damage or minimal damage to the natural nail surface on which the nail enhancement is present. For example, a nail enhancement is considered to be removable when during the removal process the nail enhancement is removed from the nail surface but its removal does not cause tearing of the nail, chipping of the nail or removal of significant chunks of the nail, A nail enhancement is considered to be removable when a residue of the multilayer nail coating remains after removal and/or minor discoloration of the nail surface results following removal. For example, white spots on the surface of the nail, or small bumps/ridges that can be remedied with a separate application of a nail strengthener, as directed, or buffing following removal of a removable nail enhancement.

The term “volatile” refers to solvent(s) which are capable of evaporating on contact with the nail surface in less than one hour, at room temperature and atmospheric pressure.

“Carboxylic acid”, as used in here refers to the group —COOH. Unless specified otherwise the term carboxylic acid embraces both the free acid and carboxylate salt. The term also encompasses compounds having multiple carboxylic acid groups thereon.

“Alkyl”, as used herein, refers to saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain, C₃-C₃₀ for branched chain), more preferably 20 or fewer carbon atoms, more preferably 12 or fewer carbon atoms. and most preferably 8 or fewer carbon atoms. In some embodiments, the chain has 1-6 carbons. Likewise, preferred cycloalkyls have from 3-10 carbon atoms in their ring structure, and more preferably have 5, 6 or 7 carbons in the ring structure. The ranges provided above are inclusive of all values between the minimum value and the maximum value. The term “alkyl” includes both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, ether, ester, carbonyl (such as a carboxyl, alkoxycarbonyl, formyl, or an acyl), thiocarbonyl (such as a thioester, thionoester, a thioacetate, or a thioformate), alkoxyl, phosphoryl, phosphate, phosphonate, a phosphinate, amino, amido, amidine, imine, cyano, isocyano, nitro, azido, sulfhydryl, alkylthio, sulfate, sulfonate, sulfamoyl, sulfonamido, sulfonyl, heterocyclyl, or an aromatic or heteroaromatic moiety.

Unless the number of carbons is otherwise specified, “lower alkyl” as used herein means an alkyl group, as defined above, but having from one to ten carbons, more preferably from one to six carbon atoms, in its backbone structure. Likewise, “lower alkenyl” and “lower alkynyl” have similar chain lengths. Preferred alkyl groups are lower alkyls. The alkyl groups may also contain one or more heteroatoms within the carbon backbone. Examples include oxygen, nitrogen, sulfur, and combinations thereof. In certain embodiments, the alkyl group contains between one and four heteroatoms.

“Alkenyl” and “Alkynyl”, as used herein, refer to unsaturated aliphatic groups containing one or more double or triple bonds analogous in length (e.g., C₂-C₃₀) and possible substitution to the alkyl groups described above.

“Aryl”, as used herein, refers to 5-, 6- and 7-membered aromatic rings. The ring may be a carbocyclic, heterocyclic, fused carbocyclic, fused heterocyclic, bicarbocyclic, or biheterocyclic ring system, optionally substituted as described above for alkyl. Broadly defined, “Ar”, as used herein, includes 5-, 6- and 7-membered single-ring aromatic groups that may include from zero to four heteroatoms. Examples include, but are not limited to, benzene, pyrrole, furan, thiophene, imidazole, oxazole, thiazole, triazole, pyrazole, pyridine, pyrazine, pyridazine and pyrimidine. Those aryl groups having heteroatoms in the ring structure may also be referred to as “heteroaryl”, “aryl heterocycles”, or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, and —CN. The term “Ar” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles, or both rings are aromatic.

“Alkylaryl”, as used herein, refers to an alkyl group substituted with an aryl group (e.g., an aromatic or heteroaromatic group).

“Heterocycle” or “heterocyclic”, as used herein, refers to a group attached via a ring carbon or nitrogen of a monocyclic or bicyclic ring containing 3-10 ring atoms, and preferably from 5-7 ring atoms, containing carbon and one to four heteroatoms each selected from non-peroxide oxygen, sulfur, and N(Y) wherein Y is absent or is H, O, (C₁₋₄) alkyl, phenyl or benzyl, and optionally containing one or more double or triple bonds, and optionally substituted with one or more substituents. The term “heterocycle” also encompasses substituted and unsubstituted heteroaryl rings. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4H-carbazolyl, carbolinyl, chromanyl, chromenyl, cinnolinyl, decahydroquinolinyl, 2H,6H-1,5,2-dithiazinyl, dihydrofuro[2,3]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. “Heteroaryl”, as used herein, refers to a monocyclic aromatic ring containing five or six ring atoms containing carbon and 1, 2, 3, or 4 heteroatoms cach selected from non-peroxide oxygen, sulfur, and N(Y) where Y is absent or is H, O, (C₁-C₂₀) alkyl, phenyl or benzyl, Non-limiting examples of heteroaryl groups include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide), quinolyl (or its N-oxide) and the like. The term “heteroaryl” can include groups of an ortho-fused bicyclic heterocycle of about eight to ten ring atoms derived therefrom, particularly a benzo-derivative or one derived by fusing a propylene, trimethylene, or tetramethylene group thereto. Examples of heteroaryl include, but are not limited to, furyl, imidazolyl, triazolyl, triazinyl, oxazoyl, isoxazoyl, thiazolyl, isothiazoyl, pyraxolyl, pyrrolyl, pyrazinyl, tetrazolyl, pyridyl (or its N-oxide), thientyl, pyrimidinyl (or its N-oxide), indolyl, isoquinolyl (or its N-oxide), quinolyl (or its N-oxide), and the like.

“Halogen”, as used herein, refers to fluorine, chlorine, bromine, or iodine.

The term “substituted,” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aryloxy, substituted aryloxy, alkylthio, substituted alkylthio, phenylthio, substituted phenylthio, arylthio, substituted arylthio, cyano, isocyano, isocyanato, substituted isocyanato, isothiocyanato, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, peptide, and polypeptide groups.

Heteroatoms, such as nitrogen, may have hydrogen substituents and/or any permissible substituents of organic compounds described herein that satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Polymer”, as used herein, refers to a molecule containing 5 or more monomer repeat units.

The term “effective period of time” or “effective amount of time” refers to the amount of time which is sufficient to cause a desired condition, effect, or outcome to occur. It is typically the minimum span of time required to effectively produce the intended result.

The term “effective amount” refers to the amount of a substance or material which is sufficient to cause a desired condition, effect, or outcome to occur. It is typically the minimum amount or quantity required to effectively produce the intended result.

Numerical ranges disclosed herein disclose individually each possible number in such range, as well as any sub-ranges and combinations of sub-ranges encompassed therein. For example, a carbon range (e.g., C₁-C₁₀) is intended to disclose individually every possible carbon value and/or sub-range encompassed within the stated range. For example, a carbon length range of C₁-C₁₀ discloses C₁, C₂, C₃, C₄, C₅, C₆, C₇, C₈, C₉, and C₁₀, as well as sub-ranges encompassed within, such as C₂-C₉, C₃-C₈, C₁-C₅, etc. Similarly, an integer value range of 1-10 discloses the individual values of 1, 2, 3, 4, 5, 6, 7, 8, 9, and 10, as well as sub-ranges encompassed within. Further, a concentration range or weight percent range, such as from 1% to 2% by weight of the formulation discloses, the individual values and fractions thereof, such as 1%, 1.1%, 1.2%, 1.32%, 1.48% etc., as well as sub-ranges encompassed within.

Use of the term “about” is intended to describe values either above or below the stated value, which the term “about” modifies, in a range of approx. +/−10%; in other instances the values may range in value either above or below the stated value in a range of approx. +/−5%. When the term “about” is used before a range of numbers (i.e., about 1-5) or before a series of numbers (i.e., about 1, 2, 3, 4, etc.) it is intended to modify both ends of the range of numbers or each of the numbers in the series, unless specified otherwise.

### II. Methods of Forming Multilayer Nail Coatings and Nail Enhancements Thereon

The nail coatings disclosed herein are multilayer structures that include at least one primer layer and at least one second, distinct layer made up of a plurality of particles that undergo a phase and/or shape transition upon exposure to a removal stimulus, such as a thermal stimulus, an electromagnetic stimulus, or a combination thereof. Such a phase and/or shape transition upon exposure to the removal stimulus renders overlying nail enhancement(s) at least partially removable, such as by detachment or delamination.

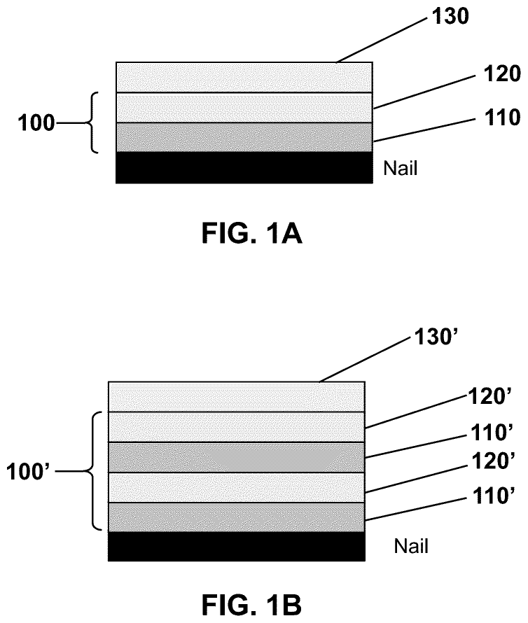

FIG. 1A shows a non-limiting illustration of a multilayer nail coating 100 including a nail primer layer 110 and a nail powder layer 120, where the multilayer nail coating is formed on a nail and a nail enhancement 130 is present on the multilayer nail coating. Nail product(s) applied onto the nail powder layer form a nail enhancement thereon.

As noted above, exposure to a removal stimulus allows for removal of the nail enhancement. Typically, such removal produces no damage or minimal damage to the natural nail surface on which the nail enhancement is present. For example, the removal procedure may not cause tearing of the nail, nor does it cause chipping of the nail or removal of significant chunks of the nail. “Minimal damage” may refer to minor discoloration, for example, some white spots on the surface of the nail, or small bumps/ridges that can be remedied with a separate application of a nail strengthener, as directed, or buffing. In some instances, a residue of the multilayer nail coating may remain after removal. This residue may also serve as a protective barrier for the natural nail, such that the removal process does not physically change, and/or does not damage, the surface of the natural nail because of the residue left behind.

Methods of forming multilayer nail coatings and removable nail enhancements thereon are described below. An exemplary method of forming such a multilayer nail coating on a nail surface includes applying a nail primer layer and applying a nail powder layer, and thereafter applying at least one or more nail products to form a nail enhancement on the multilayer nail coating, as explained below.

In one non-limiting instance, a method of forming a multilayer nail coating and a nail enhancement thereon including the steps of:
(a) applying a nail primer layer to the nail;
(b) contacting the primer layer with a nail powder composition to form a nail powder layer thereon;
(c) optionally removing any excess of the nail powder composition, such as by tapping, brushing, wiping, and/or other physical means, from the coated nail;
(d) optionally exposing the nail primer layer coated with the nail powder layer to a stimulus, such as light or heat, for a sufficient period of time to induce at least partial curing of the nail primer layer;
(e) applying one or more nail products to the multilayer nail coating formed from steps (a)-(d) to form the nail enhancement thereon;
wherein the nail powder composition comprises:
   a plurality of particles wherein at least a portion of the particles can undergo a phase and/or shape transition upon exposure to a removal stimulus, such as a thermal stimulus, an electromagnetic stimulus, or a combination thereof.

The nail powder composition generally contains a plurality of particles, and at least a portion of the particles undergo a phase and/or shape transition upon exposure to a removal stimulus, such as a thermal stimulus, an electromagnetic stimulus, or a combination thereof.

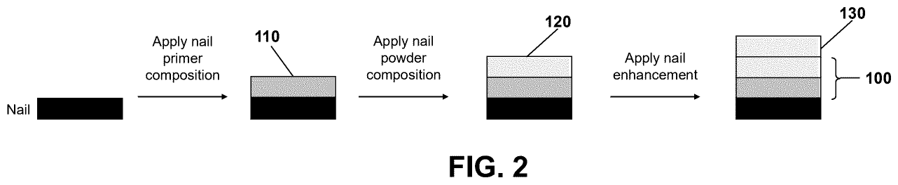

FIG. 2 shows a non-limiting scheme of a method of forming a multilayer nail coating and a nail enhancement thereon.

In some instances, during step (b) the nail powder composition is used to form the nail powder layer over the entirety or substantially the entirety of the nail primer layer. In some other instances, during step (b) the nail powder composition forms a nail powder layer over at least about 80%, 85%, 90%, 95%, 99%, or more of the total surface area of the nail primer layer that is exposed to the nail powder composition.

In some instances, steps (a)-(d) are repeated at least twice to form a stack of alternating layers of nail primer layers and nail powder layers where the final top layer, onto which the one or more nail products are applied in step (e), is a nail powder layer. For example, such a stack may include: a first nail primer layer (applied directly to a nail), a first nail powder layer, a second nail primer layer (applied on top of the first nail powder layer), and a second nail powder layer, where the second nail powder layer forms the top layer. For instance, FIG. 1B shows a non-limiting illustration of a multilayer nail coating 100′ including two nail primer layers 110′ and two nail powder layers 120′, where the multilayer nail coating, which forms a stack, is on the surface of a nail and a nail enhancement 130′ is present on the multilayer nail coating.

In some instances, the method further includes a step of partially curing or fully curing the nail primer layer following step (a) and prior to step (b). Partially curing refers to at least about 10%, 20%, 30%, 40%, 50%, 60%, 70%, 80%, 90%, 95%, 99%, or more of the monomers deposited from the nail primer composition undergoing at least one reaction, such as initiation or propagation, to produce oligomers or polymers. The reaction may occur by exposing the nail primer layer to a stimulus, such as light or heat, for a sufficient period of time to induce curing of the nail primer layer. In such instances, the cured or partially cured nail primer layer retains the ability to have a nail powder layer formed thereon when contacted with the nail powder composition in step (b).

In some instances of the methods, after the nail powder composition is applied on the nail primer layer, i.e., after step (b) and/or (c), the multilayer nail coating becomes non-adhesive and/or is not tacky (i. e, the surface is tack-free) prior to application of the one or more nail products that form the nail enhancement. The nail powder layer formed onto the cured or partially cured nail primer layer can render the top surface of the multilayer coating non-adhesive and/or not tacky. The tack or tackiness of a surface can be evaluated by known methods, such as a finger test which is a qualitative test where a finger is pressed against the surface and then lifted off. The tack or tackiness of the surface is assessed based on how much force is required to remove the finger and how much, if any, of the surface component(s) stick to the finger. The higher the force needed to remove the finger from the surface, the greater tackiness is indicated. Other more quantitative tests such as ASTM standard D6195 can be used to quantify tackiness through a loop tack strength test, which measures the force required to separate a loop of material at a specified speed after being brought into contact with a specified area of a standard surface. For example, forces less than 10, 9, 8, 7, 6, 5, 4, 3, 2, 1 N, or can be considered non-tacky based on the size of the sample used to form the loop.

In some instances, the method includes a step of applying or forming a protective layer on the nail powder layer prior to applying the one or more nail products thereon during step (e). Such a protective layer may be formed by applying a nail protective composition onto the nail powder layer prior to step (e). As a non-limiting example, a suitable nail protective composition can be the same as and/or include the nail primer composition. The nail protective layer acts to reduce or eliminate damage to the nail powder layer where, for instance, the nail protective layer may prevent or reduce loss of, or substantial loss of, and/or damage to the plurality of particles from the nail powder layer which would otherwise be caused by the application of the nail product(s) thereon in the absence of a protective layer.

In some instances of the methods, step (d) includes exposing the multilayer nail coating to an effective amount of the stimulus, which is a curing stimulus, where the curing stimulus is sufficient to induce partial or complete curing of the nail primer layer having the nail powder layer coated on top. In some instances, the curing stimulus is an electromagnetic stimulus, such as light, optionally ultraviolet light, visible light, sunlight, or a combination thereof. In some instances, the nail product is exposed to the curing stimulus for a time period ranging from about 1 second to 20 minutes, about 10 seconds to 5 minutes, or about 15 seconds to 90 seconds, as well as individual values or sub-ranges contained within the aforementioned ranges. In some instances, the stimulus is a chemical stimulus, for example, using an activator solution to accelerate solidification of the multilayer nail coating.

In some instances, the one or more nail products formed on the multilayer nail coating are able to be removed by applying a removal stimulus for a sufficient period of time, such as a time period in a range from about 1 second to 120 minutes, 10 seconds to 45 minutes, 15 seconds to 20 minutes, 30 seconds to 15 minutes, 1 minute to 10 minutes, 2 minutes to 5 minutes, 5 minutes to 15 minutes, 5 minutes to 10 minutes, 10 minutes to 15 minutes, or 15 minutes to 20 minutes, as well as individual values or sub-ranges contained within the aforementioned ranges. In some instances, the primer layer remains attached or substantially attached to the nail after removing the one or more nail enhancements following application of the removal stimulus. “Substantially attached” refers to at least about 50%, 55%, 60%, 65%, 70%, 75%, 80%, 85%, 90%, 95%, 99%, or more of the primer layer remaining following application of the removal stimulus. In other instances, the primer layer is at least partially or fully removed after applying the removal stimulus.

The subject can be a mammal, such as a human. The nail to be coated can be, for example, a fingernail or a toenail. In other instances, the subject can be an avian or a reptile.

In some instances of the methods, the nail products are nail polishes, nail gels, a hybrid gel, a builder gel, an acrylic, a nail dip powder system, artificial nails (such as acrylics), or combinations thereof. The nail products used can include a base coat, a color coat, a builder coat, a powder, an activator, acrylics, and/or a top coat. Suitable nail products include commercially available nail polishes, nail gels, a hybrid gel, a builder gel, an acrylic, a nail dip powder system, and artificial nails (such as acrylics).

In some instances, the nail powder composition is applied during step (b) by brush coating, dip coating, spray coating, or powder coating. In some instances, the nail primer layer and the nail powder composition are applied at least two or more times before applying the nail product(s) (by repeating steps (a) through (d) two or more times) before applying the nail product(s) in step (e). In some instances, the primer and/or the nail powder composition and/or the nail coating is applied two or more times, optionally sequentially, and/or under, in between, and/or over the one or more nail products.

### A. Nail Primer Layer

The nail primer layer is formed from a cosmetically acceptable nail primer composition. In some instances, the nail primer layer is applied to the nail by applying a nail primer composition to the nail, where the nail primer composition includes a mixture of one or more monomers and optionally one or more crosslinkers; or the nail primer composition includes a partially polymerized mixture of one or more monomers and optionally one or more crosslinkers; or the primer layer includes a polymer. In some other instances, the nail primer composition includes a mixture of monomers wherein at least a portion of the monomers each include at least one cyclic ring comprising a disulfide or polysulfide bond therein, such as a 1,2-dithiolane, asparagusic acid, methyl asparagusic acid, α-lipoic acid, esters of α-lipoic acid (such as methyl lipoate and ethyl lipoate), amides of α-lipoic acid, multifunctional derivatives of α-lipoic acid (for example, a tris(dithiolane) formed by esterifying glycerin and α-lipoic acid), and mixtures thereof. In certain instances, the nail primer composition includes α-lipoic acid. In some instances, the one or more monomers include one or more cyclic rings including at least one disulfide or polysulfide bond or oligomers/polymers derived therefrom are present in a concentration ranging from about 0.1 to 100 wt. %, 1 to 90 wt. %, 2 to 80 wt. %, 3 to 70 wt. %, 4 to 60 wt. %, 5 to 50 wt. %, 6 to 40 wt. %, 7 to 30 wt. %, 8 to 20 wt. %, 9 to 20 wt. %, or 10 to 20 wt. % of the total weight of the nail primer composition before removal of any volatile solvent(s), as well as individual concentration values or sub-ranges contained within.

In some instances, the nail primer layer composition also includes one or more co-reactive vinyl monomers selected from acrylates, multifunctional acrylates (such as polyethylene glycol (200) diacrylate, polypropylene glycol (400) diacrylate, polytetramethylene glycol (650) diacrylate, tricyclodecane dimethanol diacrylate, ethoxylated bisphenol A diacrylate, ethoxylated trimethylolpropane triacrylate, ethoxylated glycerin triacrylate, pentaerythritol tri- or tetraacrylate, di(trimethylolpropane) tetraacrylate, dipentaerythritol polyacrylate, polypentaerythritol polyacrylate), diacrylates, triacrylates (such as trimethylolpropane triacrylate), tetraacrylates, pentaacrylates, hexaacrylates, methacrylates, dimethacrylates, trimethacrylates, tetramethacrylates, pentamethacrylates, hexamethacrylates, bis and tris(2-acryloxyethyl)isocyanurate, acrylamides, vinyl acetates, mono-alkenes, bis-alkenes, tris-alkenes, tetrakis-alkenes, pentakis-alkenes, and mixtures thereof. In some instances, the one or more co-reactive vinyl monomers are present in a concentration ranging from about 0.1 to 10 wt. %, 0.1 to 5 wt. %, 0.1 to 2.5 wt. %, 0.1 to 1 wt. %, or 0.3 to 3 wt. % of the total weight of the nail primer composition before removal of any volatile solvent(s), as well as individual concentration values or sub-ranges contained within.

In some instances, the nail primer composition includes a reactive cyanoacrylate selected from methyl 2-cyanoacrylate, ethyl 2-cyanoacrylate, n-butyl cyanoacrylate, hexyl cyanoacrylate, isobutyl cyanoacrylate, lauryl cyanoacrylate, 2-octyl cyanoacrylate, and mixtures thereof. In some instances, the sum of all reactive cyanoacrylate monomers is present in a concentration ranging from about 0.1 to 100 wt. % or 50 to 99 wt. % of the total weight of the nail primer composition before polymerization and/or removal of any volatile solvent, as well as individual concentration values or sub-ranges contained within.

In some instances, the nail primer composition includes one or more monomers that can undergo thiol-ene polymerization selected from a multifunctional thiol and a multifunctional alkene or multifunctional alkyne, or mixture thereof. In some instances, the reactive thiols and alkene monomers are present in a concentration ranging from about 0.1 to 100 wt. %, 1 to 100 wt. %, 5 to 100 wt. %, 10 to 100 wt. %, or 20 to 95 wt. % of the total weight of the nail primer composition before polymerization and/or removal of any volatile solvent, as well as individual concentration values or sub-ranges contained within.

In some instances, the nail primer composition includes reactive epoxides selected from one or more multifunctional epoxides and one or more multifunctional alcohols or amines, and mixtures thereof. In some instances, the reactive epoxide monomers are present in a concentration ranging from about 0.1 to 100 wt. %, 1 to 95 wt. %, 5 to 95 wt. %, 10 to 95 wt. %, 20 to 80 wt. %, or 30 to 70 wt % of the total weight of the nail primer composition before polymerization and/or removal of any volatile solvent, as well as individual concentration values or sub-ranges contained within.

In some instances, the nail primer layer is a gel product, such as a UV nail gel, including one or more photocurable acrylate and/or methacrylate and/or acrylamide and/or methacrylamide monomers, and crosslinkers. In some instances, the photocurable acrylate and/or methacrylate and/or acrylamide and/or methacrylamide monomers and crosslinkers are each independently present in a concentration ranging from about 0.1 to 100 wt. %, 1 to 99 wt. %, 2 to 99 wt. %, 5 to 99 wt. %, or 10 to 99 wt. % of the total weight of the nail primer layer composition before polymerization and/or removal of any volatile solvent, as well as individual concentration values or sub-ranges contained within.

In some instances, the nail primer layer coated with the nail powder composition is exposed to ultraviolet, visible, infrared radiation, and/or exposed to an elevated temperature (i.e., a thermal stimulus) during step (d) sufficient to induce the at least partial curing of the nail primer layer.

It is understood that the nail primer layer applied in step (a) is formed by applying a nail primer layer composition to the nail surface, where the nail primer composition can include the aforementioned monomers, crosslinkers, co-monomers, and other reactive components described above. In some instances, the nail primer compositions are provided and/or stored in a container which reduces or blocks exposure of light (such as ultraviolet light) to the nail primer composition contained within.

### i. Cosmetically Acceptable Excipients for Nail Primer Compositions

The nail primer compositions may include one or more cosmetically acceptable excipients as described in “Polish College: The Basics—An introduction to common nail polish ingredients.” by Doug Schoon, Dr. Vivian, B. Valenty, and Paul Brys Jun. 1, 2008) which is incorporated in relevant part herein.

Without limitation, exemplary cosmetically acceptable excipients include: humectants, emollients, oils, moisturizers, vitamins, and/or fragrances. Other suitable excipients include plasticizers. Suitable plasticizers can make the compositions more flexible and increase the durability of the nail coating, such as dibutyl phthalate (DBP) and camphor.

In some instances, the one or more cosmetically acceptable excipients are present in a concentration ranging from about 0.001 to 99 wt. % of the total weight of the nail primer composition, as well as individual concentration values or sub-ranges contained within.

### ii. Cosmetically Acceptable Solvents, Carriers, and Diluents for Nail Primer Compositions

Suitable solvents and carriers help the spreadability of nail primer compositions and/or keep the ingredients dissolved in the composition during application, but evaporate after the composition has been applied to the nail. Non-limiting examples of suitable solvents and carriers, other than water, include acetone; esters such as ethyl acetate, propyl acetate, and butyl acetate; linear and branched alcohols, such as ethanol, propanol, isopropanol, hexanol, and the like; aromatic alcohols, such as benzyl alcohol, cyclohexanol, and the like; saturated Cn to Co fatty alcohols, such as lauryl alcohol, myristyl alcohol, cetyl alcohol, stearyl alcohol, behenyl alcohol, and the like. In some instances, non-aqueous or hydrophobic auxiliary solvents can be used to prepare compositions that are substantially water-free products, such as nail lacquers. The solvents and/or carriers have a suitable volatility level such that they evaporate quickly, such as within 5 minutes, 4 minutes, 3 minutes, 2 minutes, 1 minute, 45 seconds, 30 seconds, 15 seconds, 10 seconds, or less following application of the nail primer composition to the nail surface.

Various diluents, which are typically non-volatile liquids, can be used and will remain in the nail primer coating after application. Examples include, but are not limited to, water, alcohols, fatty alcohols, polyols, low molecular weight polymers (polymers with weight average molecular weights below about 10,000 g/mol such as dimethicone, poly(ethylene glycol), acrylate copolymers, and the like), and mixtures thereof. Non-limiting examples of non-aqueous diluents include silicones, and silicone derivatives, such as dimethicone, and the like; natural and synthetic oils and waxes, such as vegetable oils, plant oils, animal oils, essential oils, mineral oils, C₁₅ to C₄₀ isoparaffins, alkyl carboxylic esters, such as glucose monoterpene esters, jojoba oil, shark liver oil, and the like. Non-limiting examples of polyols include polyhydroxy alcohols, such as glycerin, propylene glycol, butylene glycol, hexylene glycol, C₂ to C₄ alkoxylated alcohols and C₂ to C₄ alkoxylated polyols, such as ethoxylated, propoxylated, and butoxylated ethers of alcohols, diols, and polyols having about 2 to about 30 carbon atoms and 1 to about 40 alkoxy units, polypropylene glycol, polybutylene glycol, and the like. Combinations of various solvents, carriers, and/or diluents are possible.

In some instances, the total concentration of all of the solvents, carriers, and/or diluents in the nail primer composition is at least 0.5% and less than about 99%, 90%. 80%, 70%, 60%, 50%, 40%, 30%, 20%, 10%, 5%, or 1% by weight relative to the total weight of nail primer composition. In some instances, the total concentration of all of the solvents, carriers, and/or diluents in the nail composition ranges from about 1% to 99% by weight relative to the total weight of nail primer composition, as well as individual values or sub-ranges contained within. Total concentration refers to the concentration of all of the solvents, carriers, diluents, active agent(s), monomers, comonomers, oligomers, polymers, and any other components that are present in the nail primer composition combined before the evaporation of any volatile solvent.

In some instances, the total concentration of the cosmetically acceptable solvents and carrier(s) in the nail primer composition ranges from about 1 wt. % to 99 wt. % of the total weight of the nail primer composition, as well as individual values or sub-ranges contained within.

### iii. Optional Photochemical And/or Thermal Radical Initiators for Nail Primer Compositions

In some instances, the nail primer compositions further include one or more photochemical and/or thermal radical initiators. In some instances, the one or more photochemical and/or thermal radical initiators are selected, without limitation, from type-1 or type-2 photoinitiators, such as benzoin ethers, benzil ketals, α-dialkoxy-acetophenones, α-hydroxy-alkylphenones, α-aminoalkyl-phenones, acyl phosphine oxides, benzophenones, thioxanthones, α-diketones such as camphorquinone and derivatives thereof, α-hydroxyketones, and/or titanocenes. In some instances, coinitiators such as amines can be used to improve the reactivity of photoinitiators like camphorquinone or derivatives thereof. In other instances, the one or more photochemical and/or thermal radical initiators are selected, without limitation, from 2,2-dimethoxy-2-phenylacetophenone, phenylbis(2,4,6-trimethylbenzoyl)phosphine oxide, diphenyl(2,4,6-trimethylbenzoyl)phosphine oxide, lithium phenyl-2,4,6-trimethylbenzoylphosphinate, bis(2,6-dimethoxybenzoyl)(2,4,4-trimethylpenty1) phosphine oxide, 2-hydroxy-2-methylpropiophenone, camphorquinone, coumarin, 1-hydroxycyclohexyl phenyl ketone, peroxides (such as benzoyl peroxide), azo initiators (such as azobisisobutyronitrile), benzophenone, ring-opening polymerization initiators (such as thiolates), ring-opening polymerization sulfonium-ion initiators, and combinations thereof. The one or more photochemical and/or thermal radical initiators may be present in a concentration ranging from about 0.0001 wt. % to 20 wt. % of the total weight of the nail primer composition, as well as individual values or sub-ranges contained within.

### iv. Optional Stabilizing Agent(s), Thiol-Capping Agent(s), And/or UV Absorbing Agent(s) for Nail Primer Compositions

In some instances, the nail primer compositions further include at least one stabilizing agent. The stabilizing agent can reduce or prevent changes in odor, appearance, composition and/or viscosity over time compared to the same composition in the absence of the stabilizing agent(s) over the same period of time, such as for a period of time ranging from at least about 3 to 24 months or 6 to 12 months, as well as individual times or sub-ranges contained within the aforementioned ranges. Without limitation, suitable stabilizing agent(s) include hydroquinone, 2,6-di-tert-butyl-4-methylphenol (butylated hydroxytoluene, BHT), 4-tert-butylcatechol (TBC), 4-methoxyphenol (MEHQ), methylethyl ketone oxime, phenylbis(2,4,6-trimethylbenzoyl)phosphine oxide, diphenyl(2,4,6-trimethylbenzoyl)phosphine oxide, gallic acid, gallic acid esters, or a combination thereof. The stabilizing agent(s) may be present in a total concentration ranging from about 0.0001 wt. % to 20 wt. % of the total weight of the nail primer composition, as well as individual values or sub-ranges contained within.

In some instances, the nail primer compositions described herein further include a thiol capping agent. The thiol capping agent can reduce or prevent changes in odor, appearance, composition and/or viscosity over time compared the same composition in the absence of the thiol capping agent(s) over the same period of time, such as for a period of at least about 3 to 24 months or 6 to 12 months, as well as individual times or sub-ranges contained within the aforementioned ranges. Without limitation, suitable thiol capping agent(s) include 2-iodoacetamide, 2-iodo-N-methylacetamide, 2-bromo-acetamide, methyl isocyanide, or 1,3-dimethyl-1H-pyrrole-2,5-dione, or a combination thereof. In some instances, the total concentration of the thiol capping agent(s) ranges from about 0.0001 wt. % to 20 wt. % of the total weight of the nail primer composition, as well as individual values or sub-ranges contained within.

In some instances, the nail primer compositions further include one or more ultraviolet absorbing agents. The ultraviolet absorbing agents can absorb light having wavelengths of up to about 385 nm, up to about 415 nm, or up to about 450 nm. In some instances, the ultraviolet absorbing agents can absorb light having wavelengths in a range from about 385 to 450 nm, as well as individual values or sub-ranges contained within the aforementioned ranges. Without limitation, the one or more ultraviolet absorbing agents can be avobenzone, zinc oxide, homosalate, octocrylene, benzotriazole and derivatives thereof, “red-shifted UV absorbers” (RUVA), 5-trifluoromethyl-2-(2-hydroxy-3-alpha-cumyl-5-tert-octylphenyl)-2H-benzotriazole (available under the trade designation “CGL-0139” from BASF, Florham Park, NJ). Other exemplary benzotriazoles can include, without limitation, 2-(2-hydroxy-3,5-di-alpha-cumylphehyl)-2H-benzotriazole, 5-chloro-2-(2-hydroxy-3-tert-butyl-5-methylphenyl)-2H-benzotriazole, 5-chloro-2-(2-hydroxy-3,5-di-tert-butylphenyl)-2H-benzotriazole, 2-(2-hydroxy-3,5-di-tert-amylphenyl)-2H-benzotriazole, 2-(2-hydroxy-3-alpha-cumyl-5-tert-octylphenyl)-2H-benzotriazole, and 2-(3-tert-butyl-2-hydroxy-5-methylphenyl)-5-chloro-2H-benzotriazole, or a combination thereof. Suitable RUVAs include 2(-4,6-diphenyl-1-3,5-triazin-2-yl)-5-hexyloxy-phenol. Other suitable UV absorbing agents include those available from BASF under the trade designations “TINUVIN 1577,” “TINUVIN 900,” “TINUVIN 1600,” and/or “TINUVIN 777.” Still other suitable UV absorbing agents are available, for example, in a polyester master batch under the trade designation “TA07-07 MB” from Sukano Polymers Corporation, Dunkin, SC. Another exemplary suitable UV absorbing agent for polymethylmethacrylate is a masterbatch available, for example, under the trade designation “TA11-10 MB01” from Sukano Polymers Corporation. Yet other suitable UV absorbing agents for polycarbonate is a masterbatch from Sukano Polymers Corporation, under the trade designations “TA28-09 MB01.” In addition, the UV absorbers can be used in combination with hindered amine light stabilizers (HALS) and anti-oxidants. Exemplary HALS include those available from BASF, under the trade designations “CHIMASSORB 944” and/or “TINUVIN 123.” Exemplary anti-oxidants include those obtained under the trade designations “IRGANOX 1010” and/or “ULTRANOX 626”, also available from BASF. Combinations of any of the aforementioned ultraviolet absorbing agents are possible. In some instances, the total concentration of the one or more ultraviolet absorbing agents ranges from about 0.0001 wt. % to 20 wt. % of the total weight of the nail primer composition, as well as individual values or sub-ranges contained within.

### v. Optional Abrasion or Scratch Resistance Additives for Nail Primer Compositions

In certain instances, the nail primer compositions further include one or more additives that increase abrasion resistance or scratch resistance of the formed nail coating. The total concentration of the one or more additives to increase abrasion or scratch resistance can range from about 0.0001 to 50 wt. % of the total weight of nail primer composition, as well as individual values or sub-ranges contained within. Without limitation, the one or more additives to increase abrasion or scratch resistance can be minerals, silicones, nanoclays, comonomers such as isobornyl acrylate, allyl diglycol carbonate, polymer-based additives (such as poly(methyl methacrylate)), polysiloxanes, polyurethanes, MoS₂, graphite, oleic acid amide, or aliphatic polyurethane acrylate, or a combination thereof.

### vi. Optional Thermal Management Additives for Nail Primer Compositions

In certain instances, the nail primer compositions further include one or more thermal management additives that can dissipate, conduct, and/or absorb a sufficient amount of thermal energy (such as when the removal stimulus is a thermal stimulus) to at least partly reduce the exposure of the nail surface to high temperatures which could damage the natural nail surface on which the nail primer layer is formed on. In some instances, use of the thermal management additive(s) can prevent the nail surface from being exposed to temperatures greater than about 35° C. to 45° C., 38° C. to 43° C., or 38° C. to 40.5° C., as well as individual values or sub-ranges contained within the aforementioned ranges. Exemplary thermal management additives can be selected, without limitation, from ceramic particles, hollow ceramic particles, polymer particles, hollow polymer particles, yttria-stabilized zirconia particles, hollow yttria-stabilized zirconia particles, mullite, alumina, cerium oxide, rare earth zirconates, rare earth oxides, metal-glass composites, or combinations thereof. In some instances, the total concentration of the one or more thermal management additives are present at ranges from about 0.0001 wt. % to 50 wt. % of the total weight of the nail primer composition, such as from about 0.0001 wt. % to 0.25 wt. %, from 0.0001 wt. % to 0.20 wt. %, from 0,001 wt. % to 15 wt. %, from 0.001 wt. % to 10 wt. %, from 0.001 wt. % to 5 wt. %, from 0.001 wt. % to 1 wt. %, from 0,01 wt. % to 10 wt. %, from 0.01 wt. % to 5 wt. %, from 0,01 wt. % to 1 wt%, from 0.1 wt. % to 10 wt. %, from 0.1 wt. % to 5 wt. %, or from 0,1 wt. % to 1 wt. %, as well as individual values or sub-ranges contained within.

### B. Nail Powder Compositions

The nail powder composition is cosmetically acceptable. Various nail powder compositions are described herein which can be used in the formation of a multilayer nail coating that enables the removability of nail enhancement(s) formed thereon upon exposure to a suitable removal stimulus, as described in the methods above. The nail powder compositions can be used to form a nail powder layer on the nail primer layer.

In some instances, a nail powder composition includes a plurality of particles, where at least a portion of the particles undergo a phase and/or shape transition upon exposure to a stimulus, such as a thermal stimulus, an electromagnetic stimulus, or a combination thereof. The nail powder composition also optionally includes one or more primary additives that improve performance, such as: (1) improved adhesion to a nail primer layer and/or improved adhesion to a layer of a nail product layer(s), (2) increased shape and/or volume change of the particles of the plurality, when exposed to the removal stimulus, and/or (3) reduced damage to the multilayer nail coating when applying additional layers of a primer or a nail product on top of a nail powder layer formed from the nail powder composition. “Improved adhesion” can refer to an increase of at least about 1 to 100% in adhesion, as compared to the same nail powder composition absent the primary additive(s), as well as individual values or sub-ranges contained within. The one or more primary additives may be selected to modify the flowability of the nail powder composition to improve the feel to the user during application as a powder and/or to reduce the amount of excess powder deposited on the nail (which would need to be brushed off). The primary additive(s) may also reduce or eliminate solid residue left on the skin around the nail. Optionally, the one or more primary additives may modify the gloss, sheen, or color of the nail powder layer, or reduce cost and the like.

In some instances, the one or more primary additives are selected from the group consisting of silicon dioxide, titanium dioxide, polymer particles (such as linear or crosslinked polymer particles), and combinations thereof. Non-limiting examples of polymer particles, which may be crosslinked, include poly(acrylates), poly(methacrylates), poly(styrenes), crosslinked poly(vinyl) derivatives, and copolymers derived therefrom. The polymer particles can be derived from emulsion polymerization, miniemulsion polymerization, or suspension polymerization, among other possible techniques used to form polymer particles having monodisperse or polydisperse size distributions with diameters in the nanometer or micrometer length scale. In some instances, the polymers formed, depending on the method of making (such as emulsion polymerization), are non-crosslinked polymer particles, crosslinked polymer particles, or mixtures thereof. The polymer particles may contain additional additives such as peroxides, amines, and other molecules that are used to induce, initiate, or catalyze a reaction in the layer(s) beneath and/or the layer(s) above the deposited powder. In some instances, the one or more primary additives may be dip powders used in commercially available dip powder systems. In some instances, the one or more primary additives present in the nail powder composition are present in a total concentration in a range from about 0.1 to 99.9 wt. % of the total weight of the nail powder composition, in a range from about 10 wt. % to 90 wt. %, 20 wt. % to 80 wt. %, or 30 wt. % to 70 wt. % of the total weight of the nail powder composition. In some instances, where at least a portion of the particles undergo a phase and/or shape transition upon exposure to a stimulus, such as a thermal stimulus, an electromagnetic stimulus, or a combination thereof, those particles that undergo the phase and/or shape transition are primary particles having secondary particles contained within.

In some instances, a nail powder composition includes a plurality of primary particles which are formed of linear or crosslinked polymer(s) having a size suitable for holding one or more secondary particles therein. In some instances, the primary particles have an average diameter of at least about 50, 75, 100, 125, 150 or more microns, or in a range from between about 50 to 150 microns, as well as individual values or subranges contained within. The primary particles exhibit no thermal expansion or substantially no thermal expansion activity when exposed to a removal stimulus, such as described above. “Substantially no thermal expansion activity” refers to less than about 5%, 4%, 3%, 2%, 1%, 0.5%, or 0.1% change in the shape and/or volume change of the particles made of the linear or crosslinked polymer(s), when exposed to the removal stimulus. The secondary particles, which are contained within the primary particles, are thermally expandable such that they exhibit an increase in shape and/or volume change when exposed to a removal stimulus, such as described above. Without limitation, the primary particles may be formed from monomers, such as acrylates, methacrylates, acrylamides, styrenes, vinyl ethers, vinyl esters, vinyl monomers, allyl monomers, lactones, lactams, ureas, isocyanates, polyester prepolymers and monomers, polyurethane prepolymers and monomers, silicones, and combinations thereof; the resulting primary polymeric particles may or may not be crosslinked depending on the synthesis conditions for forming primary particles thereof. Without limitation, the secondary particles, which are thermally expandable, may be thermally expandable microspheres, such as those described in detail below. Methods of making polymeric particles that contain other particles therein are known. For the instances described having secondary particles contained within primary particles, the resulting nail composition is understood to be in a powder form and formed of the discrete particles making up the composition.

In some instances, at least some of the particles in the plurality of particles are thermally expandable microspheres. Such thermally expandable microspheres can be prepared by the encapsulation of a gaseous component in a suitable thermoplastic polymer shell. The gaseous or blowing component expands inside the polymer shell upon the application of a thermal energy stimulus for an effective period of time. The gaseous or blowing component can include, without limitation, one or more gas agents such as C₃-C₇ alkane gases, such as butane, isobutane, propane, heptane and the like, fluorinated alkanes, and mixtures thereof.

Without limitation, exemplary suitable commercially available thermally expandable microspheres are sold under the name EXPANCEL® MICROSPHERES (Nouryon, Inc), and are formed of a thermoplastic vinyl resin polymer shell and core composed of alkane gas. EXPANCEL® MICROSPHERES offer a variety of microspheres with different expansion temperatures and properties. This includes, without limitation, EXPANCEL®551 DE, EXPANCEL® 920 DE, and EXPANCEL® 031 DU. Other commercially available thermally expandable microspheres include: DUALITE® (Henkel) such as DUALITE® M 6001 AE and DUALITE® M 7000 AE; MICROPEARL® (Matsumoto Yushi-Seiyaku Co., Ltd.) such as MICROPEARL® F 100D and MICROPEARL® F 80D; SPHERICEL® (Potters Industries), such as SPHERICEL® 60P40 and SPHERICEL® 110P8; and SUNSPHERE® (Sunjin Chemical) such as SUNSPHERE® H-33 and SUNSPHERE® H-51,

Combinations of different types of thermally expandable microsphere particles and such primary additives may also be included in the nail powder compositions.

The particles in the nail powder compositions can have any suitable average diameter. In some instances, the plurality of particles has an average particle diameter of between about 1 to 100 micrometers, 1 to 90 micrometers, 1 to 80 micrometers, 1 to 70 micrometers, 1 to 60 micrometers, 1 to 50 micrometers, 1 to 40 micrometers, 1 to 30 micrometers, 1 to 20 micrometers, or 1 to 10 micrometers, as well as individual values or sub-ranges contained within the aforementioned ranges. In some instances, the plurality of particles has an average particle diameter ranging from about 40 to about 80 micrometers, as well as individual values or sub-ranges contained within the aforementioned range. The average particle diameter can be measured by various known methods, such as by dynamic light scattering using a commercial particle size analyzer, laser diffraction, and microscopy techniques.

In some instances, the particles are conditioned, treated, and/or modified to decrease the temperature needed to activate the phase and/or shape transition or change the effective period of time required to induce the thermally induced phase and/or shape transition of the particles, as compared to the same particles in the absence of the conditioning, treatment, and/or modification.

In some instances, the particles, such as thermally expandable microspheres, undergo at least a partial volume expansion that is sufficient to cause a change in adhesion of the nail enhancement to the natural nail and/or nail coating at temperatures between about 30 to 80°C., 35 to 75° C., 40 to 65° C., 45 to 60° C., 45° C. to 55° C., 50° C. to 60° C., 50° C. to 55° C., 45 to 50° C., or 55° C. to 60° C., as well as individual values or sub-ranges contained within the aforementioned ranges. It is understood that there can be an interplay between time and temperature to cause sufficient expansion, meaning that in some instances longer times may be used for expansion at lower temperatures and shorter times may be used for expansion at higher temperatures. The nail powder composition may be selected to undergo sufficient expansion in the aforementioned ranges of temperatures in time periods ranging from about 1 second to 120 minutes, 10 seconds to 45 minutes, 15 seconds to 20 minutes, 30 seconds to 15 minutes, 1 minute to 10 minutes, 2 minutes to 5 minutes, 5 minutes to 15 minutes, 5 minutes to 10 minutes, 10 minutes to 15 minutes, or 15 minutes to 20 minutes, as well as individual values or sub-ranges contained within the aforementioned ranges. In some cases, the longer time required to cause a change in adhesion at lower temperatures may be an advantage for users as it acts to protect them against unwanted de-adhesion during daily activities such as washing hands in warm or hot water or while taking a shower.

In some instances, the nail powder composition is dry and substantially free of any solvents, carriers, or other volatile species or liquids.

In other instances, the plurality of particles is suspended (i.e., forms a suspension) in a cosmetically acceptable excipient such as a volatile solvent, a non-volatile diluent, or a combination thereof. In such instances, it is understood that the volatile solvent will evaporate to produce the nail powder layer without damaging or substantially damaging the underlying nail primer layer. Without limitation, exemplary volatile solvents can be selected from acetone, ethyl acetate, butyl acetate, ethanol, isopropanol, and combinations thereof. In some instances, the volatile solvent is ethanol or isopropanol.

In other instances, the nail powder composition is provided as a slurry (also referred to as a “wet cake”) of the plurality of particles in one or more solvents or carriers or a combination of at least one volatile solvent and at least one non-volatile diluent. In some instances, such a slurry includes the plurality of particles in water, ethanol, isopropanol, glycerin, propylene glycol, or combinations thereof, or the like. In some instances, the slurry includes about 1 to 99 wt. % of the particles, 10 to 99 wt. % of particles, 20 to 99 wt. % of particles, 30 to 99 wt. % of particles, 40 to 99 wt. % of particles, 50 to 99 wt. % of particles, 60 to 99 wt. % of particles, 70 to 99 wt. % of particles, 80 to 99 wt. % of particles, 90 to 99 wt. % of particles, or 65 to 80 wt. % of particles, as well as individual values or sub-ranges contained within the aforementioned ranges.

In some instances, the plurality of particles is present in a concentration in a range from about 0.1 to 100 wt. %, 0.1 to 75 wt. %, 0.1 to 50 wt. %, 0.1 to 25 wt. %, 0.1 to 20 wt. %, 0.1 to 15 wt. %, 0.1 to 10 wt. %, 0.1 to 5 wt. %, or 0.1 to 1 wt. % of the nail powder composition, as well as individual values or sub-ranges contained within the aforementioned ranges.

In some instances, the plurality of particles are conditioned, treated, or modified before being applied to the nail primer layer to decrease the temperature needed to induce or activate the phase and/or shape transition, reduce the period of time required to induce the thermally induced phase and/or shape transition at a given temperature, and/or increase the change in shape or size when exposed to the stimulus, as compared to the plurality of particles in the absence of conditioning, treatment, or modification. For example, this pre-treatment might involve heating the particles at a specific temperature in an oven. In other instances, heating at a specified temperature may be performed in a closed chamber with a controlled atmosphere, such as in the presence of solvent vapor, for example, using glycerin, propylene glycol, alcohols, or the like. Conditioning by thermal annealing may also be performed by submersing the plurality of particles in a solvent or mixture of solvents, optionally containing other solutes such as sodium chloride, potassium chloride, calcium carbonate, or the like, or a combination thereof. The solvent may or may not be evaporated leaving the other solutes in the powder composition.

In some instances, treatment of the plurality of particles is performed after applying the plurality of particles as a powder to the primer layer but before applying any additional nail primer layers or nail product layers on top. In a non-limiting example, this treatment might involve brushing a non-reactive solvent, such as glycerin, onto the as-applied nail powder layer using a twist pen nail applicator, as sold, for example, by E-lishine. In some cases, multiple treatments are applied for example after the brushing treatment, a wiping step can be included to remove excess glycerin.

In some instances, the plurality of particles includes one or more secondary additives therein, where the one or more secondary additives can lower the volume expansion temperature and/or volume expansion time and/or increase the stimulus-induced change in volume of the thermally expandable microspheres, as compared to the thermally expandable microspheres without the one or more secondary additives therein. In some instances, the secondary additive includes at least one plasticizing solvent having a boiling point of at least 100° C. at 1 atm of pressure, such as water, glycerin, dimethylsulfoxide, sulfolane, Cyrene™, lactones (valerolactone, dodecalactone, undecalactone, decalactone, and the like), and eucalyptol. In some other instances, the one or more secondary additives are salts such as sodium chloride or potassium chloride, or combinations thereof.

The handling, storage and shipping of a solid or dry formulation of expandable microspheres or a mixture of expandable microspheres and one or more solid additives can be advantageous to maintaining the efficacy and performance of the nail powder composition for providing removal of nail enhancements according to the methods described herein. In some instances, forming a dispersion of the expandable microspheres in a liquid or viscoelastic matrix may result in a decrease in efficacy and performance over time with the extent of expansion decreasing to a point where the nail enhancement removal proceeds with the same difficulty as when no expandable microspheres are used. Accordingly, in some instances, it can be advantageous to avoid exposing the expandable microspheres or mixture thereof to liquids, such as ethyl acetate, that can dissolve or plasticize the microsphere shell and damage the plurality of particles over time.

### i. Optional Pigment/dye Additives for Nail Powder Compositions

The nail powder compositions may include one or more cosmetically acceptable pigments or dyes as further additives. In some instances, the nail powder compositions may exclude such pigments or dyes. Optionally, when dyes or pigments are present in the nail powder composition, they may or may not modify the color, hue, and/or appearance of the final coating formed after applying all of the overlying layers. Combinations of naturally occurring and manufactured pigments/dyes can be blended together to create a desired shade or hue.

In some instances, the pigments or dyes may be coated or uncoated pigments, water-soluble dyes, or liposoluble dyes, or a mixture thereof. The term “pigments” means white or colored, mineral or organic particles, which are insoluble in an aqueous medium, and which are intended to color and/or opacify the nail powder composition and/or a coating formed thereof.

Suitable mineral pigments that may be used in the nail powder compositions include, but are not limited to, zirconium oxide or cerium oxide, zinc oxide, iron oxide (black, yellow or red) or chromium oxide, manganese violet, ultramarine blue, chromium hydrate and ferric blue, titanium dioxide, and metal powders, for instance aluminum powder and copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS. In some instances, the size of the pigment that is useful may generally be greater than about 100 nanometers and may range to 10 micrometers, from 200 nanometers to 5 micrometers, or from 300 nanometers to 1 micrometers. In some instances, the pigments have a size characterized by a D[50] greater than 100 nanometers and possibly ranging up to 10 micrometers, from 200 nanometers to 5 micrometers, or from 300 nanometers to 1 micrometer, where D[50] represents the maximum size that 50% by volume of the particles have. The sizes can be measured by static light scattering using a commercial MasterSizer 30000 particle size analyzer from Malvern, which makes it possible to determine the particle size distribution of all of the particles over a wide range, which may extend from 0.01 micrometers to 1000 micrometers. The data are processed on the basis of the standard Mie scattering theory, which is suitable for size distributions ranging from submicrometer to multimicrometer length scales and allows an “effective” particle diameter to be determined. See, e.g., Van de Hulst, H. C., Light Scattering by Small Particles, Chapters 9 and 10. Wiley, New York, 1957. Individual size values or sub-ranges of the aforementioned ranges are possible.

In some instances, the nail powder compositions also include nacres. The term “nacres” is understood as meaning colored particles of any form, which may or may not be iridescent, notably produced by certain mollusks in their shell, or alternatively synthesized, and which have a color effect via optical interference. The nacres may be nacreous pigments, such as titanium mica coated with an iron oxide, titanium mica coated with bismuth oxychloride, titanium mica coated with chromium oxide, titanium mica coated with an organic dye, and also nacreous pigments based on bismuth oxychloride. They may also be mica particles, at the surface of which are superposed at least two successive layers of metal oxides and/or of organic dyes. Without limitation, suitable examples of nacres include natural mica covered with titanium oxide, with iron oxide, with natural pigment or with bismuth oxychloride. The nacres may have a yellow, pink, red, bronze, orange, brown, gold and/or coppery color or tint.

In some instances, the nail powder compositions include one or more organic pigments or dyes. The term “organic pigment” refers to any pigment that satisfies the definition in Ullmann's Encyclopedia of Industrial Chemistry, “Organic Pigments” (7^{th} Ed.) (2012). The organic pigment can be a nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane, and/or quinophthalone compounds. In certain instances, the organic pigment(s) may be, for example, carmine, carbon black, aniline black, melanin, azo yellow, quinacridone, phthalocyanine blue, sorghum red, the blue pigments codified in the Color Index under the references Cl 42090, 69800, 69825, 73000, 74100 and 74160, the yellow pigments codified in the Color Index under the references Cl 11680, 11710, 15985, 19140, 20040, 21100, 21108, 47000 and 47005, the green pigments codified in the Color Index under the references Cl 61565, 61570 and 74260, the orange pigments codified in the Color Index under the references Cl 11725, 15510, 45370 and 71105, the red pigments codified in the Color Index under the references Cl 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and/or the pigments obtained by oxidative polymerization of indolic or phenolic derivatives, Suitable organic dyes, include, without limit, cochineal carmine, D&C Red 21 (Cl 45 380), D&C Orange 5 (Cl 45 370), D&C Red 27 (Cl 45 410), D&C Orange 10 (Cl 45 425), D&C Red 3 (Cl 45 430), D&C Red 4 (Cl 15 510), D&C Red 33 (Cl 17 200), D&C Yellow 5 (Cl 19 140), D&C Yellow 6 (Cl 15 985), D&C Green 5 (Cl 61 570), D&C Yellow 10 (Cl 77 002), D&C Green 3 (Cl 42 053), and/or D&C Blue 1 (Cl 42 090).

Suitable pigments may be in the form of composite pigments, which are composed of particles including a mineral core at least partially covered with an organic pigment and at least one binder for fixing the organic pigments to the core.

In some other instances, the pigment may also be a lake pigment. A lake pigment is made by precipitating a dye with an inert binder, or mordant, usually a metallic salt; i.e., an insolubilized dye adsorbed onto insoluble substrates/particles, the assembly thus obtained remaining insoluble during use. The inorganic substrates onto which the dyes are adsorbed can be, for example, alumina, silica, calcium sodium borosilicate, or calcium aluminum borosilicate and aluminum.

As noted above, the dyes can be water-soluble dyes. Water soluble dyes refer to any natural or synthetic, generally organic compound, which is soluble in an aqueous phase or water-miscible solvents and which is capable of imparting color, Non-limiting examples of suitable water-soluble dyes include, for instance, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, and/or FDC Blue 1. Suitable natural water-soluble dyes include anthocyanins.

As noted above, the dyes can also be liposoluble dyes, which refer to any natural or synthetic, generally organic compound, that is soluble in an oily phase or in solvents that are miscible with the oily phase, and that is capable of imparting color. Non-limiting examples of suitable liposoluble dyes include, for instance, DC Red 17, DC Red 21, DC Red 27, DC Green 6, DC Yellow 11, DC Violet 2, DC Orange 5, Sudan red and Sudan brown. Other natural liposoluble dyes, can include, without limit, carotenes, for instance 3-carotene, a-carotene and lycopene; quinoline yellow; xanthophylls such as astaxanthin, antheraxanthin, citranaxanthin, cryptoxanthin, canthaxanthin, diatomoxanthin, flavoxanthin, fucoxanthin, lutein, rhodoxanthin, rubixanthin, siphonaxanthin, violaxanthin, zeaxanthin; annatto; curcumin; quinizarin (ceres green BB, D&C green No. 6, 01 61565, 1,4-di-p-toluidinoanthraquinone, green No. 202, quinazine green SS), and/or chlorophylls.

In some instances, the pigments or dyes in the nail powder compositions are present at a total concentration of less than about 50%, 40%, 30%, 20%, 10%, 5%, 4%, 3%, 2%, 1%, 0.5%, 0.1%, or 0.01% by weight relative to the total weight of nail powder composition prior to application to a nail surface.

### ii. Optional Abrasion or Scratch Resistance Additives for Nail Powder Compositions

In certain instances, the nail powder compositions further include one or more additives that increase abrasion resistance or scratch resistance of a multilayer nail coating formed using such powder compositions. The one or more additives that increase abrasion or scratch resistance can be present in a total concentration ranging from about 0.0001 to 50 wt. %, 0.0001 to 25 wt. %, 0.0001 to 20 wt. %, 0.0001 to 15 wt. %, 0.0001 to 10 wt. %, 0.0001 to 5 wt. %, or 0.0001 to 1 wt. % of the total weight of nail powder composition, as well as individual values or sub-ranges contained within. Without limitation, the one or more additives to increase abrasion or scratch resistance can be minerals, silicones, nanoclays, polymer-based additives (such as poly(methyl methacrylate)), polysiloxanes, polyurethanes, MoS₂, graphite, oleic acid amide, or aliphatic polyurethane acrylate, or a combination thereof.

### C. Multilayer Nail Coatings and Properties Thereof

The multilayer nail coating, formed from at least one nail primer layer and nail powder layer, does not negatively affect or impact the nail enhancements formed thereon. For instance, following application of the one or more nail products onto the multilayer nail coating, the resulting nail enhancement has the same look and feel as the same nail product, such as a gel and dip, when applied directly onto a nail surface (i.e. in absence of the multilayer nail coating). Accordingly, the presence of the multilayer nail coating allows for facile removal of the nail enhancement when desired, but does not affect the other properties and performance of a nail enhancement.

In some instances, the multilayer nail coating, and nail enhancement(s) thereon, remain intact when rinsed or submerged in water or a water-based solution at a temperature range from about 0° C. to 50° C. when washed using soap and water. In some other instances, the multilayer nail coating, and nail enhancement(s) thereon, remain intact and attached or substantially attached to the nail when rinsed or submerged in water or a water-based solutions containing salt, surfactants, and/or an alcohol (such as ethanol) at temperatures ranging from about 0° C. to 60° C., 0° C. to 50° C., 0° C. to 45° C., or 0° C. to 40° C. The multilayer nail coating, and nail enhancement(s) thereon, can be evaluated, for example, using microscopic imaging, profilometry, and/or abrasion testing.

In certain instances, the multilayer nail coating formed can impart one or more desirable properties on a nail surface, as compared to the same nail surface prior to formation of the multilayer nail coating thereon.

In some instances, the multilayer nail coating strengthens and/or hardens the nail of a subject, as compared to the nail prior to formation of the multilayer nail coating. Hardness can be determined, for example, using a pencil hardness test or a Vickers or Knoop indenter. Additional suitable testing systems include, but are not limited to, the Sward Rocker and the Tukon Microhardness Tester. Other suitable techniques are described in the article by M. L. Schlossman, J. Soc. Cosmet. Chemists, 1981, 32, pages 43-52. In some instances, the strength, scratch resistance, and/or hardness of the nail is increased by at least about 1 to 100%, as well as individual values or sub-ranges disclosed within. In some instances, the strength, scratch resistance, and/or hardness of the nail is increased by at least about 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

In some instances, the multilayer nail coating increases resistance to wear, abrasion, and/or tearing, and/or improves the robustness, look, and/or feel of the nail of a subject, as compared to the nail prior to application of the multilayer nail coating thereon. The increased resistance to wear and abrasion can be assessed using a tribometer or by nail metrology (such as flexural, tensile, and/or tearing tests). In some instances, the resistance to wear, abrasion, and/or tearing of the nail is increased by at least about 1 to 100%, as well as individual values or sub-ranges disclosed within. In some instances, the resistance to wear, abrasion, and/or tearing of the nail is increased by at least about 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

Look, feel, and/or robustness can be assessed by visual or tactile inspection and may include a reduction of ridges and the filling in of defects present in the nail prior to formation of the nail coating thereon. In some instances, the improvement in look and/or feel may also be assessed by microscopic imaging, profilometry, and/or abrasion testing, which can compare the nail's surface quality before and after formation of the multilayer nail coating.

### III. Methods of Removing Nail Enhancements

The multilayer nail coatings formed by the methods described herein can be used in various applications in the nail care industry. The nail enhancements overlying the multilayer nail coatings are at least partially removable by exposure to a suitable removal stimulus, as described in the removal methods detailed below.

In one non-limiting instance, a method of removing a nail enhancement formed by any of the methods described herein includes the steps of:
(a′) applying a removal stimulus for an effective amount of time to a multilayer nail coating and nail enhancement thereon;
wherein the removal stimulus is applied for an effective period of time to induce an effective amount of the plurality of particles of the nail powder layer of the multilayer nail coating to undergo a thermally induced phase and/or shape transition causing debonding, delamination, detachment, and/or a change in density that renders the nail enhancement at least partially removable, as compared to prior to the application of the removal stimulus. In some instances, the nail enhancement may or may not spontaneously and/or fully detach after applying the removal stimulus for a sufficient amount of time. When detachment is not spontaneous after applying the removal stimulus for a sufficient time period, the adhesion of the nail enhancement to the multilayer nail coating is significantly changed such that the nail enhancement can be removed with greater ease, for example, by prying along the edges with tools commonly used in the field of nail care.

In some instances, the removal stimulus is a thermal stimulus, photochemical stimulus, photothermal stimulus, or electromagnetic stimulus, or a combination thereof. In some instances, the removal stimulus is a thermal stimulus and the effective period of time ranges from about 1 second to 120 minutes, 10 seconds to 45 minutes, 15 seconds to 20 minutes, 30 seconds to 15 minutes, 1 minute to 10 minutes, 2 minutes to 5 minutes, 5 minutes to 15 minutes, 5 minutes to 10 minutes, 10 minutes to 15 minutes, or 15 minutes to 20 minutes. In some instances, the removal stimulus is a thermal stimulus having a temperature in a range from about 30 to 80° C., 35 to 75 ° C., 40 to 65° C., 45 to 60 ° C., 45° C. to 55° C., 50 ° C. to 60° C., 50° C. to 55° C., 45° C. to 50° C., or 55° C. to 60° C., as well as individual values and sub-ranges contained within the aforementioned ranges.

In some instances, the method further includes applying an induction responsive nail heating composition directly on top of the nail enhancement prior to step (a′) that can be used to generate a thermal stimulus. In such instances, the induction responsive nail heating composition can be used to form a liquid, solid, or viscoelastic layer that includes a metal, metal alloy, metal oxide, semiconductor, ceramic, or other magnetically inductive responsive material that generates heat when exposed to an alternating magnetic field. In some instances, the metal, metal alloy, metal oxide, semiconductor, ceramic, or other induction responsive material is a suspension in a liquid, solid, or viscoelastic material. In some instances, the metal, metal alloy, metal oxide, semiconductor, ceramic, or induction responsive material is Fe₃O₄, Fe₂O₃, XyZwFe_{3-y-w}O₄ (where X, Z can independently be Zn, Ni, Mn, Co, or Cu; and where 0≤y≤3, 0≤w≤3, and 0≤y+w≤3), a divalent transition metal, an alkali earth metal, ferrites, carbon, carbon fiber, silicium, graphene, graphite, steel, stainless steel, mild steel, iron, tin, tungsten, copper, copper alloys, brass, aluminum, chrome, nickel, nickel alloys, cobalt, platinum, silver, or gold, or a combination thereof.

An induction heating apparatus, such as an induction heating coil, can be used to apply an alternating magnetic field to the induction heating nail composition layer on the nail enhancement. The induction heating apparatus can operate at a frequency from about 10 kHz to 5 MHz, or a subrange therein, for a period of time ranging from about 1 second to 120 minutes, 10 seconds to 45 minutes, 15 seconds to 20 minutes, 30 seconds to 15 minutes, 1 minute to 10 minutes, 2 minutes to 5 minutes, 5 minutes to 15 minutes, 5 minutes to 10 minutes, 10 minutes to 15 minutes, or 15 minutes to 20 minutes during step (a′).

In some instances, the methods of removing described above refer to the complete and total removal of the nail enhancement from the natural nail and/or multilayer nail coating due the application of the removal stimulus. In some other instances, the methods of removing described above refer to the near total removal of the nail enhancement from the natural nail and/or multilayer nail coating surface due the application of the removal stimulus, where “near total” refers to removal of at least about 75% to 99.9% of the nail enhancement and/or multilayer nail coating from the nail surface, as well as individual values and sub-ranges contained within the aforementioned range. In yet other instances, the methods of removing described above facilitate or improve the ability to remove the nail enhancement from the natural nail and/or nail coating, where the application of the removal stimulus allows for at least about 1% to 99.9% of the nail enhancement and/or multilayer nail coating to be removed from the natural nail and/or multilayer nail coating, such as by peeling, nicking, and/or scratching of the nail enhancement, as well as individual values and sub-ranges contained within the aforementioned range. In some instances, the methods of removing improve the ability to remove the nail enhancement from the natural nail and/or multilayer nail coating by at least about 5% to 100%, as compared to a natural nail coated with a primer layer having a nail product thereon but which excludes a nail powder layer, or as compared to a natural nail coated with the nail product alone, as well as individual values and sub-ranges contained within the aforementioned range. In most or all cases, the multilayer nail coating, nail primer layer, and/or nail powder layer may remain substantially intact after removing the nail enhancement, or any one of the multilayer nail coating, nail primer layer, and/or nail powder layer may be partially or fully removed along with the nail enhancement.

### IV. Kits

Kits useful for practicing the methods described above, and compositions used therein, are also disclosed. For example, kits are provided for nail care applications, such as forming multilayer nail coatings and applying nail enhancements (such as commercial nail products) thereon which are removable on-demand by way of application of a suitable removal stimulus.

In one non-limiting instance, a kit includes: a nail primer composition; a nail powder composition; optionally a brush applicator, such as an E-lishine 3 mL Transparent Twist Pens with Brush Tip, to apply a treatment to the as-deposited powder layer; and optionally one or more nail products (such as in the form of a strengthener, a base coat, a color coat, a builder coat, a top coat, UV resistant gloves or mittens, and/or a UV blocker). In some instances, the kit further includes a treatment device for applying secondary additive(s), as described above, to the nail powder composition that can, for example, improve the removal performance of the nail enhancement when a removal stimulus is applied. Nail primer compositions for forming a nail primer layer, and nail powder compositions for forming a nail powder layer, are described in detail above. Suitable nail products include, without limitation, commercially available nail products, including nail polishes, nail gels, hybrid gels, builder gels, acrylics, nail dip powder systems, and artificial nails (such as acrylics).

In some instances, kits can further include one or more of the following:
(i) a nail trimmer, cuticle pusher, a nail buffer, and/or a nail file;
(ii) a UV lamp, LED lamp, visible light source, or infrared source;
(iii) UV-blocking formulation and/or protective gloves, such as a skin conditioning lotion with UV blocking additives therein and/or UV blocking gloves, respectively;
(iv) a cuticle oil;
(v) nail art (e.g., stickers, gems, freehand designs);
(vi) a nail heating device or system, which can provide a thermal or electromagnetic stimulus to the top surface of a nail enhancement, such as sticky and optionally disposable resistive heating pads, a power supply, and optionally an embedded or auxiliary temperature sensor, proportional-integral-derivative (PID) controller for regulating the temperature of the heating pads, and/or an induction heating apparatus or induction heating coil;
(vii) an induction nail heating composition (as described above); and/or
(viii) instructions for use.

In some instances, the UV or LED lamp is a commercially available UV/LED nail lamp (such as a SUNUV SUN2C, LKES8) or a light-emitting diode source purchased from Thorlabs, Inc. In some instances, the UV or LED lamp provides a narrow emission wavelength which is centered around 365 nm, 385 nm, 405 nm, and 415 nm. In some instances, the narrow emission wavelength is centered in a range of about 365 to 415 nm. In some instances, the narrow emission wavelength has a majority of the intensity (greater than about 50%) emitted at wavelengths in the visible range above 400 nm in wavelength.

### EXAMPLES

### Example 1: Thermal Removal of a Nail Gel Enhancement Formed on a Multilayer Nail Coating

### Materials and Methods

A nail primer composition including lipoic acid (15 wt %) and phenylbis(2,4,6-trimethylbenzoyl)phosphine oxide (1 wt %) in ethyl acetate was coated onto a glass slide and air-dried for 1 minute to form a nail primer layer.

Onto the solid but uncured primer layer was coated a layer of 031 DU 40 thermally expandable microspheres (Nouryon) by dipping the primer layer attached to the glass slide into a solid powder of the 031 DU 40 thermally expandable microspheres. Excess powder was gently removed by tapping the glass slide on a table yielding a thin layer of the solid microspheres on top of the primer layer that was visually separate from the underlying primer layer, forming a tack-free surface.

The microsphere-coated primer layer was cured by exposure to a commercially available UV/LED nail lamp (SUNUV SUN2C) for 30 seconds to form a multilayer nail coating.

A gel nail enhancement system was then applied (DND DC nail gel) on the multilayer nail coating per the manufacturer's instructions: a base coat layer was applied on top of the primer layer and cured, followed by two color coats, and a top coat. Each layer of nail gel was cured under a UV/LED nail lamp for 90 seconds before adding the next coat. Thus, a nail enhancement was formed on the multilayer nail coating on the glass slide.

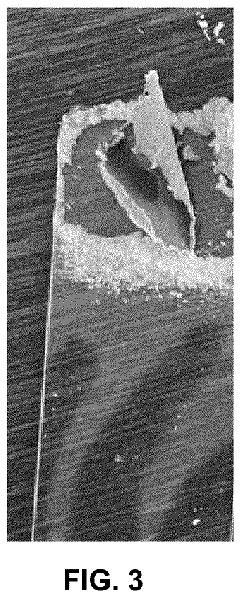

In order to test removal of the nail enhancement, the glass slide was placed on a hot plate (T=110° C.) for 30 seconds, which caused the nail enhancement to completely detach from the glass slide (see FIG. 3).

### Example 2: Thermal Removal of a Nail Dip System Enhancement Formed on a Multilayer Nail Coating

### Materials and Methods

A nail primer composition including lipoic acid (15 wt %) and phenylbis(2,4,6-trimethylbenzoyl)phosphine oxide (1 wt %) in ethyl acetate was coated onto a glass slide and air-dried for 1 minute to form a nail primer layer.

Onto the solid but uncured primer layer was coated a layer of 031 DU 40 thermally expandable microspheres (Nouryon) by dipping the primer layer attached to the glass slide into a solid powder of the 031 DU 40 thermally expandable microspheres. Excess powder was gently removed by tapping the glass slide on a table yielding a thin layer of the solid microspheres on top of the primer layer that was visually separate from the underlying primer layer, forming a tack-free surface.

The microsphere-coated primer layer was cured by exposure to a commercially available UV/LED nail lamp (SUNUV SUN2C) for 30 seconds to form a multilayer nail coating.

Onto this nail coating was applied a Kiara Sky dip powder system following the manufacturer's instructions. A base coat was painted onto the nail coating and dipped into a natural powder. This process of applying base coat and dipping into powder was repeated 2 times using a colored powder, followed by once more using the clear powder. A seal protectant was added after the color coats followed by a top coat, which was allowed to solidify for 10 minutes.

After heating the sample on a hot plate at 110° C., expansion of the microspheres caused detachment of the dip powder nail enhancement from the nail coating and the glass slide.

### Example 3: Thermal Removal of a Nail Dip System Enhancement formed on a Multilayer Nail Coating containing a Lipoic Acid Primer Layer and a 1:9 Mixed Layer of Thermally Expandable Microspheres and Dip Powder Microparticles

### Materials and Methods

A nail primer composition including lipoic acid (15 wt %) and phenylbis(2,4,6-trimethylbenzoyl)phosphine oxide (1 wt %) in ethyl acetate was coated onto a glass slide and air-dried for 1 minute to form a nail primer layer.

Onto the solid but uncured primer layer was coated a mixture of 031 DU 40 thermally expandable microspheres (Nouryon) and clear Kiara Sky dip powder (1:9, microspheres: dip powder) by dipping the glass slide into the powder mixture. Excess powder was gently removed by tapping the glass slide on a table yielding a thin layer of solid microsphere mixture on top of the primer layer that was visually separate from the underlying primer layer, forming a tack-free surface.

The powder-coated primer layer was cured by exposure to a commercially available UV/LED nail lamp (SUNUV SUN2C) for 30 seconds to form a multilayer nail coating. This process was repeated one additional time, resulting in a total of two powder layers and two primer layers.

A dip nail enhancement system was then applied (Kiara Sky dip system) per the manufacturer's instructions: a base coat layer was applied on top of the primer layer and dipped into Kiara Sky dip powder and repeated two additional time. A seal protectant was added after the color coats followed by a top coat, which was allowed to solidify for 10 minutes. This resulted in a dip nail enhancement formed on the nail coating on the glass slide.

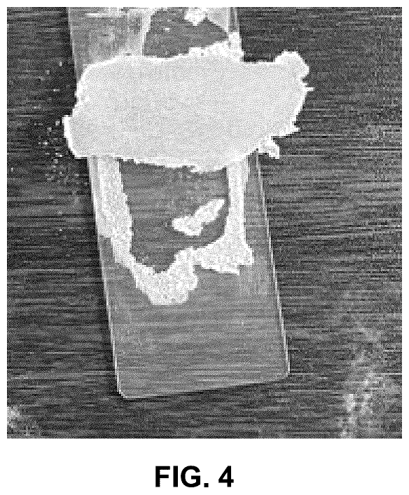

In order to test removal of the nail enhancement, the glass slide was placed on a hot plate (T=110 ° C.) for 30 seconds, which caused the nail enhancement to completely detach from the glass slide (see FIG. 4).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific instances of the invention described herein. Such equivalents are intended to be encompassed by the following claims.

1. A method of forming a multilayer nail coating and a nail enhancement thereon comprising the steps of:
(a) applying a nail primer layer to the nail;
(b) contacting the nail primer layer with a nail powder composition to form a nail powder layer thereon;
(c) optionally removing any excess of the nail powder composition, such as by tapping, brushing, wiping, and/or other physical means, from the coated nail;
(d) optionally exposing the nail primer layer coated with the nail powder layer to a stimulus, such as light or heat, for a sufficient period of time to induce at least partial curing of the nail primer layer;
(e) applying one or more nail products to the multilayer nail coating formed from steps (a)-(d) to form the nail enhancement thereon;
wherein the nail powder composition comprises:
a plurality of particles wherein at least a portion of the particles can undergo a phase and/or shape transition upon exposure to a removal stimulus, such as a thermal stimulus, an electromagnetic stimulus, or a combination thereof.

2. The method of claim 1, further comprising a step of applying one or more treatments to improve detachment performance and/or adhesion prior to step (e).

3. The method of claim 1, wherein steps (a)-(d) are repeated two or more times.

4. The method of claim 1, wherein the particles of the plurality are thermally expandable microspheres.

5. The method of claim 1, wherein the thermally expandable microspheres undergo at least partial volume expansion at temperatures between about 30 to 80° C., or between 35 to 75° C., or between 40 to 65° C., or between 45 to 60° C., or between 45° C. to 55° C., or between 50° C. to 60° C., or between 50° C. to 55° C., or between 45° C. to 50° C., or between 55° C. to 60° C.

6. The method of claim 1 wherein the plurality of particles is present in a concentration in a range from about 0.1 to 99.9 wt. % of the nail powder composition.

7-9. (canceled)

10. The method of claim 1, wherein the plurality of particles is deposited from a suspension in a volatile solvent, a non-volatile diluent, or combination thereof.

11. The method of claim 1, wherein step (d) is performed and comprises exposing the multilayer nail coating to an effective amount of the stimulus, which is a curing stimulus,
wherein the curing stimulus is sufficient to induce partial or complete curing of the nail primer layer having the nail powder layer coated on top.

12-14. (canceled)

15. The method of claim 1, wherein the one or more nail products are selected from the group consisting of nail polish, nail gel, hybrid gel, builder gel, acrylics, nail dip powder systems, artificial nails, and combinations thereof.

16. The method of claim 1, wherein the nail powder composition is applied by brush coating, dip coating, spray coating, or powder coating.

17-18. (canceled)

19. The method of claim 1, wherein the nail primer layer is applied to the nail by applying a nail primer composition that comprises a mixture of one or more monomers and optionally one or more crosslinkers; or the nail primer composition is a partially polymerized mixture of one or more monomers and optionally one or more crosslinkers; or the primer layer is a polymer.

20. The method of claim 19, wherein the nail primer composition comprises a mixture of monomers wherein at least a portion of the monomers each include at least one cyclic ring comprising a disulfide or polysulfide bond therein optionally selected from a 1,2-dithiolane, asparagusic acid, methyl asparagusic acid, α-lipoic acid, esters of a-lipoic acid amides of α-lipoic acid, multifunctional derivatives of α-lipoic acid, and mixtures thereof.

21. The method of claim 19, wherein the nail primer composition comprises one or more co-reactive vinyl monomers selected from the group consisting of acrylates, multifunctional acrylates, diacrylates, triacrylates tetraacrylates, pentaacrylates, hexaacrylates, methacrylates, dimethacrylates, trimethacrylates, tetramethacrylates, pentamethacrylates, hexamethacrylates, bis and tris(2-acryloxyethyl)isocyanurate, acrylamides, vinyl acetates, mono-alkenes, bis-alkenes, tris-alkenes, tetrakis-alkenes, pentakis-alkenes, and mixtures thereof; or wherein the one or more co-reactive monomers comprise more than five alkene, acrylate, or alkyne groups; or
wherein the nail primer composition comprises a reactive cyanoacrylate selected from the group consisting of methyl 2-cyanoacrylate, ethyl 2-cyanoacrylate, n-butyl cyanoacrylate, hexyl cyanoacrylate, isobutyl cyanoacrylate, lauryl cyanoacrylate, 2-octyl cyanoacrylate, and mixtures thereof.

22-24. (canceled)

25. The method of claim 19, wherein the nail primer composition is a nail gel comprising one or more photocurable acrylate, methacrylate, acrylamide, and/or methacrylamide monomers, and/or crosslinkers.

26. The method of claim 1, wherein the nail primer layer with the nail powder thereon is exposed to ultraviolet, visible, infrared radiation, and/or an elevated temperature during step (d), when performed, sufficient to induce the at least partial curing of the nail primer layer.

27. The method of claim 1, wherein following step (a) and prior to step (b) the method further comprises a step of exposing the nail primer layer with a curing stimulus, for a sufficient period of time to induce at least partial curing of the nail primer layer.

28-29. (canceled)

30. A method of removing the nail enhancement present on the multilayer nail coating formed by the method of claim 1, comprising the steps of:
(a′) applying a removal stimulus for an effective amount of time to the multilayer nail coating and nail enhancement thereon;
wherein the removal stimulus is applied for an effective period of time to induce an effective amount of the plurality of particles of the nail powder layer to undergo a thermally induced phase and/or shape transition causing debonding, delamination, detachment, and/or a change in density that renders the nail enhancement at least partially removable, as compared to prior to the application of the removal stimulus.

31. The method of claim 30, wherein the removal stimulus is applied for a sufficient period of time in a range from about 1 second to 120 minutes, 10 seconds to 45 minutes, 15 seconds to 20 minutes, 30 seconds to 15 minutes, 1 minute to 10 minutes, 2 minutes to 5 minutes, 5 minutes to 15 minutes, 5 minutes to 10 minutes, 10 minutes to 15 minutes, or 15 minutes to 20 minutes.

32. The method of claim 30, wherein the removal stimulus is selected from the group consisting of a thermal stimulus, photochemical stimulus, photothermal stimulus, electromagnetic stimulus, and combinations thereof.

33. The method of claim 30, wherein the removal stimulus is a thermal stimulus and the effective period of time is a time ranging from about 1 second to 120 minutes, 10 seconds to 45 minutes, 15 seconds to 20 minutes, 30 seconds to 15 minutes, 1 minute to 10 minutes, 2 minutes to 5 minutes, 5 minutes to 15 minutes, 5 minutes to 10 minutes, 10 minutes to 15 minutes, or 15 minutes to 20 minutes.

34. The method of claim 30 wherein the removal stimulus is a thermal stimulus having a temperature in a range from about 30 to 80° C., 35 to 75° C., 40 to 65° C,, 45 to 60° C., 45° C. to 55° C., 50° C. to 60° C., 50° C. to 55° C., 45° C. to 50° C., or 55° C. to 60° C.

35-39. (canceled)

40. A nail powder composition comprising:
a plurality of particles wherein at least a portion of the particles can undergo a phase and/or shape transition upon exposure to a removal stimulus, such as a thermal stimulus, an electromagnetic stimulus, or a combination thereof; and
one or more primary additives that improve performance, such as improved adhesion to a nail primer layer and/or adhesion to a nail product, increase shape and/or volume change of the particles of the plurality when exposed to the removal stimulus, and/or reduce damage to the multilayer nail coating when applying additional layers of a nail primer or a nail product over a nail powder layer formed from the nail powder composition.

41. The nail powder composition of claim 40, wherein at least portion of the particles of the plurality are thermally expandable microspheres.

42. The nail powder composition of claim 41, wherein the thermally expandable microspheres undergo at least partial volume expansion at temperatures between about 30 to 80° C., 35 to 75° C., 40 to 65° C., 45 to 60° C., 45° C. to 55° C., 50° C. to 60° C., 50° C. to 55° C., 45° C. to 50° C., or 55° C. to 60° C. in a time ranging from about 1 second to 30 minutes, 10 seconds to 25 minutes, 15 seconds to 20 minutes, 30 seconds to 15 minutes, 1 minute to 10 minutes, 2 minutes to 5 minutes, 5 minutes to 15 minutes, 5 minutes to 10 minutes, or 10 minutes to 15 minutes.

43. The nail powder composition of claim 40, wherein the plurality of particles is present in a concentration in a range from about 0.1 to 99.9 wt. % of the nail powder composition.

44. The nail powder composition of claim 40, wherein the one or more primary additives are selected from the group consisting of silicon dioxide, titanium dioxide, polymer particles (such as linear or crosslinked polymer particles), and combinations thereof.

45-50. (canceled)

51. The nail powder composition of claim 40, wherein the nail primer compositions further comprise one or more thermal management additives that can dissipate, conduct, and/or absorb thermal energy.

52. (canceled)

53. A kit comprising the nail powder composition of claim 40.

54. The kit of claim 53, further comprising:
a treatment device for applying one or more secondary additives to the nail powder composition that improve the removal performance of a nail enhancement by a removal stimulus; or
one or more heating devices or systems used to apply a removal stimulus; or
one or more nail products; or
one or more UV-blocking compositions and/or UV-blocking protective gloves.

55-57. (canceled)
